(19) United States
(12) Patent Application Publication
(10) Pub. No.: US 2024/0016628 A1
(43) Pub. Date: Jan. 18, 2024

(54) SPACER BLOCKS, SYSTEMS AND METHODS FOR TRIALING KNEE JOINT

(71) Applicant: Tim Yoko, Granger, IN (US)

(72) Inventor: Tim Yoko, Granger, IN (US)

(21) Appl. No.: 18/221,153

(22) Filed: Jul. 12, 2023

Related U.S. Application Data
(60) Provisional application No. 63/388,862, filed on Jul. 13, 2022.

(51) Int. Cl. A61F 2/46

(52) U.S. Cl. CPC A61F 2/4684; A61F 2002/3069

(57) ABSTRACT

In one aspect, a provisional tibial prosthesis system is shown and described. The provisional tibial prosthesis system optionally includes any one or combination of: a tibial component, a base component and a shim. The tibial component can have a rail that defines a pocket and a distal surface configured to seat on a resected proximal surface of a tibia. The base component can be configured to seat within the pocket of the tibial component when positioned thereon. When so positioned with the pocket, the base component can have a substantially flat proximal surface and a substantially flat distal surface. The shim can have a feature configured to couple with a second feature of the base component when disposed thereon. The shim can have a substantially flat proximal surface.

### CLAIM OF PRIORITY

This application claims the benefit of U.S. Provisional Patent Application Ser. No. 63/388,862, filed on Jul. 13, 2022, the benefit of priority of which is claimed hereby, and which is incorporated by reference herein in its entirety.

### BACKGROUND

Orthopedic procedures and prostheses are commonly utilized to repair and/or replace damaged bone and tissue in the human body. For example, a knee arthroplasty can be used to restore natural knee function by repairing damaged or diseased articular surfaces of the femur and/or tibia. An incision is made into the knee joint to expose the bones comprising the joint. Cut guides are used to guide the removal of the articular surfaces that are to be replaced. Prostheses are used to replicate the articular surfaces. Knee prostheses can include a femoral component implanted on the distal end of the femur, which articulates with a tibial component implanted on the proximal end of a tibia to replicate the function of a healthy natural knee. Various types of arthroplasties are known including a total knee arthroplasty, where all of the articulating compartments of the joint are repaired with prosthetic components.

Provisional knee prosthesis systems, including a plurality of provisional components, can be positioned on a distal end of a femur or a proximal end of a tibia to allow a surgeon to test and appropriately fit a permanent knee prosthesis system within a patient. During surgery, the surgeon can remove and replace various of the provisional components based upon fit and other criteria to arrive at an appropriate configuration of the permanent knee prosthesis system.

An implant revision surgery is a process by which an existing implant is removed to be replaced by a new implant. However, due to the bond between the implant to be removed and the bone, the bone is often damaged during implant removal. As a result, the subsequent positioning and installation of a replacement implant may lack precision due to damaged bone surfaces. For instance, in knee revision surgery, machining of the bone surfaces using conventional cutting blocks may lack precision as conventional bone landmarks used for defining the orientation of the cutting block may be altered or removed during the removal of the implant.

Use of provisional knee prosthesis systems during an implant revision surgery can be difficult as soft tissue, bone and other landmarks may be removed. This makes determining a frame of reference to achieve appropriate sizing and joint kinematics challenging.

### Overview

This disclosure pertains generally to provisional tibial prostheses, systems, and methods, including one or more provisional tibial components that can collectively be used to replicate permanent (or final) tibial components. When used provisionally, the tibial prostheses, systems, and methods disclosed herein can assist in determining a proper configuration of a permanent tibial prosthesis system that is designed to replace all or a portion of a knee joint. The present tibial prostheses, systems, and methods can be used in conjunction with one or more permanent tibial prosthesis systems.

The present inventor recognizes, among other things, that existing provisional implants, systems, and methods can be overly complex to mount within the joint space and/or difficult to utilize when judging joint gap(s) such as the flexion gap, the extension gap and medial-lateral positioning and in making other spatial determinations used in replicating knee joint kinematics and determining an appropriate joint line. In particular, some provisional systems utilize shims or inserts that replicate articular surfaces of the joint. Although shaped articular surface(s) can be useful for determining range of motion, the shape of these surfaces (concave, dish and otherwise shaped) can be difficult to reference off and/or can be unnecessarily constraining. Furthermore, the present inventor recognizes that many provisional systems when used in a revision knee procedure require a complete resection of the femoral box prior to checking flexion and extension gaps. Additionally, the present inventor recognizes in the revision knee procedure, a relative lack of landmarks (bone, soft tissue, etc.) can make it difficult to properly judge and set femoral rotation. This difficulty can be exacerbated with some provisional systems, which have articular surface shape and/or features that can interfere with determining and setting femoral rotation.

Considering the above, the present inventor proposes a provisional system that utilizes an easy to install and easy to reference base component (and if needed shim). In particular, a proximal surface of the base component or shim, which with other systems would otherwise be shaped for articulation with a femoral provisional component or implant, can be substantially flat or can have a known constant angulation. This substantially flat surface or known angulation for the shim or base component can be easy for the surgeon to work with to visualize and reference off of Such referencing can include visual based referencing (e.g., the surgeon visually inspecting gap sizes, spacing, etc.) and/or instrument based referencing (e.g., using a calipers, a cut guide or other tool that contacts or otherwise uses the proximal surface or other part of the shim or base component as a reference. Furthermore, the base component can have features that allow for easy installation on a tibial provisional component, tibial implant or for coupling with a shim. The base component merely needs to be set in place on the tibial provisional component or implant by the surgeon. The system can include a plurality of shims that can be used with the base component (e.g., stacked on top thereof) as desired. The shim that is utilized and base component and need not be removed prior to arriving at an appropriate configuration for the permanent tibial prosthesis system. Removal of the shim and base component can be easily accomplished.

To further illustrate the apparatuses, systems and methods disclosed herein, the following non-limiting examples (referred to below as aspects and/or techniques) are provided:

In some aspects, the techniques described herein relate to a provisional tibial prosthesis system optionally including any one or combination of: a tibial component having a rail that defines a pocket and a distal surface configured to seat on a resected proximal surface of a tibia; a base component configured to seat within the pocket of the tibial component when positioned thereon, wherein when so positioned with the pocket, the base component has a substantially flat proximal surface and a substantially flat distal surface; and a shim having a feature configured to couple with a second feature of the base component when disposed thereon, wherein the shim has a substantially flat proximal surface.

In some aspects, the techniques described herein relate to a system, wherein optionally neither the proximal surface of the base component nor the proximal surface of the shim is configured to articulate with a femoral prosthesis.

In some aspects, the techniques described herein relate to a system, wherein optionally the feature of the shim is configured to mate with and be complementary to the second feature of the base component.

In some aspects, the techniques described herein relate to a system, wherein optionally the distal surface of the base component and the proximal surface of the base component have a substantially same shape and size so as to be reversable in orientation between a right knee and a left knee of a patient.

In some aspects, the techniques described herein relate to a system, further optionally including a third feature of the base component having an identical configuration with the second feature of the base component, wherein the third feature is on a distal side of the base component and the second feature is on a proximal side of the base component.

In some aspects, the techniques described herein relate to a system, wherein optionally the feature of the shim includes a boss with a dovetail and the second feature of the base component includes a notch with a corresponding dovetail.

In some aspects, the techniques described herein relate to a system, wherein optionally the shim is one of a plurality of shims each having a different proximal-distal thickness from one another.

In some aspects, the techniques described herein relate to a system, wherein optionally any one of the plurality of shims is stackable on the base component by sliding the feature of one of the plurality of shims anterior-posterior into the second feature of the base component.

In some aspects, the techniques described herein relate to a system, wherein optionally the feature and the second feature are complementary mating features configured to couple with one another in a mating engagement when the shim is placed atop the proximal surface of the base component.

In some aspects, the techniques described herein relate to a system, wherein optionally the feature includes a male projection and the second feature includes a complimentary female recess in the base component.

In some aspects, the techniques described herein relate to a system, wherein optionally each of the shim and the base component have a removal feature to aid in removing the shim and the base component from a knee joint.

In some aspects, the techniques described herein relate to a system, wherein optionally a periphery of the base component is configured to seat against an interior of the rail of the tibial component.

In some aspects, the techniques described herein relate to a system, wherein optionally the tibial component includes one of a provisional component or a permanent implant.

In some aspects, the techniques described herein relate to a system, further optionally including a femoral cut guide configured to reference one of the proximal surface of the base component or the proximal surface of the shim when making a resection of a femur.

In some aspects, the techniques described herein relate to a system, wherein optionally the base component with the substantially flat distal surface rests on a substantially flat proximal surface of the tibial component within the pocket, and wherein the base component has no feature to couple with the tibial component when so seated on the substantially flat proximal surface of the tibial component but is held in position within the pocket atop the substantially flat proximal surface of the tibial component by gravity and/or a compressive force.

In some aspects, the techniques described herein relate to a provisional prosthesis system optionally including any one or combination of: a tibial component having a distal surface configured to seat on a resected proximal surface of a tibia; a base component configured to mount on a tibial component and having a substantially flat proximal surface and a substantially flat distal surface; and a set of shims, each of the set of shims configured to couple with the base component and having a different proximal-distal thickness, wherein each of the set of shims has a substantially flat proximal surface and a substantially flat distal surface.

In some aspects, the techniques described herein relate to a system, wherein optionally a periphery of the base component is configured to seat against an interior of a rail of the tibial component.

In some aspects, the techniques described herein relate to a system, wherein optionally the shim has a feature and the base component has a second, wherein the feature and the second feature are complementary mating features configured to couple with one another in a mating engagement when the shim is placed atop the proximal surface of the base component.

In some aspects, the techniques described herein relate to a system, further optionally including a third feature of the base component having an identical configuration with the second feature of the base component, wherein the distal surface of the base component and the proximal surface of the base component have a substantially same shape and size so as to be reversable in orientation between a right knee and a left knee of a patient, and wherein the third feature is on a distal side of the base component and the second feature is on a proximal side of the base component.

In some aspects, the techniques described herein relate to a method of using a provisional tibial prosthesis system during knee arthroplasty procedure, the method optionally including any one or any combination of: mounting a tibial component on a resected proximal surface of a tibia; setting a base component with a substantially flat proximal surface on a proximal surface of the tibial component without mechanically coupling the base component to the tibial component with use of one or more coupling feature; moving a knee joint through a range of motion; and referencing at least one of a flexion gap and an extension gap relative to the substantially flat proximal surface of the base component.

In some aspects, the techniques described herein relate to a method, further optionally including: coupling a shim having a substantially flat proximal surface to the base component such that the shim is set atop the substantially flat proximal surface of the base component; moving the knee joint through a range of motion; and referencing at least one of a flexion gap and an extension gap relative to the substantially flat proximal surface of the shim.

In some aspects, the techniques described herein relate to a method, further optionally including: setting a femoral cut guide atop one of the substantially flat proximal surface of the shim or the substantially flat proximal surface of the base component; and performing a resection of a femur with the aid of the femoral cut guide.

These and other examples and features of the present apparatuses, systems and methods will be set forth in part in the following Detailed Description. This Overview is intended to provide non-limiting examples of the present subject matter—it is not intended to provide an exclusive or exhaustive explanation. The Detailed Description below is included to provide further information about the present apparatuses, systems and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1 and 2 illustrate knee joint structures providing suitable environments in which a provisional tibial prosthesis system in accordance with an example of the present application can be utilized.

FIG. 3A is a perspective view of a provisional tibial component that can be part of a provisional system in accordance with at least one example of this disclosure.

FIG. 3B is a plan view of a proximal side of the provisional tibial component of FIG. 3A.

FIGS. 4A and 4B are plan views of a proximal side and an anterior side of a base component of the provisional system in accordance with at least one example of this disclosure.

FIGS. 5A and 5B are plan view of a proximal side and an anterior side of a shim of the provisional system in accordance with at least one example of this disclosure.

FIG. 6 shows a system of shims of different proximal-distal thicknesses that are useable with the base component of FIGS. 4A and 4B in accordance with at least one example of this disclosure.

FIG. 7 shows a tibial provisional assembly created with some of the components of the system of FIGS. 3-5B in accordance with at least one example of this disclosure.

FIG. 8 shows a femoral cut guide coupled to and referencing the tibial provisional assembly of FIG. 7 in accordance with at least one example of this disclosure.

### DETAILED DESCRIPTION

The present application relates to provisional prostheses, systems, and methods. The systems, for example, can comprise a provisional tibial prosthesis system that includes a provisional tibial component or permanent tibial implant, a base component, and one or more shims (sometime called shim components herein). The shim is optional and need not be utilized in some circumstances as dictated by the patient's joint line and joint kinematics. The shim (if used) can be used as a height varying spacer between the base component and a femoral component. The shim can be added to the base component of the provisional tibial prosthesis system to alter a spacing between the provisional tibial prosthesis system and the femoral component. This adjustment can be used to simulate a permanent tibial prosthesis system, visualize joint gaps, ascertain spacing and perform other tasks such as femoral resection(s).

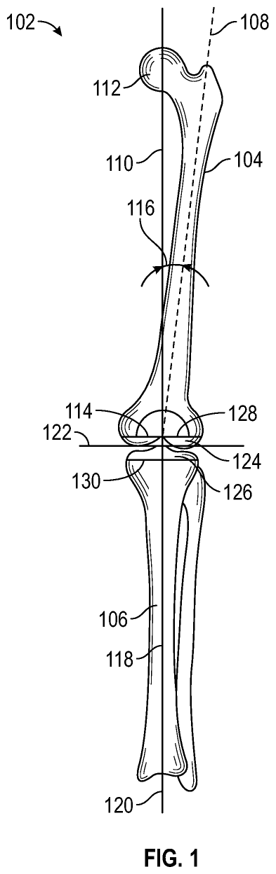

To better understand knee joint replacement procedures, it can be helpful to understand the relationship of bones and bone cuts that can be made to orient various provisional and permanent prosthesis components within a knee joint. FIGS. 1 and 2 illustrate several features of knee joint structures and orientations. In FIG. 1, a frontal view of a lower limb 102, including a femur 104 and a tibia 106, is shown to illustrate various lower limb axes. The femur 104 has an anatomic axis 108 that coincides generally with its intramedullary canal. The femur 104 also has a mechanical axis 110, or load axis, running from the center of a femoral head 112 to the center of a knee joint 114. The angle 116 extending between these two axes varies among the patient population, but is generally on the order of between 5-7 degrees, inclusive. Like the femur 104, the tibia 106 also has an anatomic axis coinciding generally with its intramedullary canal. The mechanical axis 118 of the tibia 106 runs from the center of the knee joint 114 to the center of an ankle region 120 and is generally collinear with its anatomic axis.

A joint line 122, about which the knee joint 114 flexes, is approximately parallel to a line through medial and lateral femoral condyles 124 and to a tibial plateau 126. Although illustrated as perpendicular in FIG. 1, the joint line 122 can extend at a varus or valgus angle relative to the mechanical axes 110 and 118 of the femur 104 and tibia 106, respectively. Normally, during a partial or total knee replacement procedure, portions of a distal end of the femur 104 or a proximal end of the tibia 106 are resected to be parallel or approximately parallel to the joint line 122, and thus perpendicular to the mechanical axes 110 and 118, as indicated at 128 and 130, respectively.

FIG. 2 illustrates a closer view of the knee joint 114 and its coordinate system, in which a medial/lateral axis 202 corresponds approximately to the joint line 122 (FIG. 1), a proximal/distal axis 204 corresponds approximately to the mechanical axes 110 and 118 (FIG. 1), and an anterior/posterior axis 206 is approximately normal to the other two axes. Position along each of these axes can be depicted by arrows, which can represent the medial/lateral 208, anterior/posterior 210, and proximal/distal 212 positioning of inserted prosthesis components. Rotation about each of these axes can also be depicted by arrows. Rotation about the proximal/distal axis 204 can correspond anatomically to external rotation of a femoral component, while rotation about the anterior/posterior axis 206 and medial/lateral axis 202 can correspond to extension plane slope and varus/valgus angle of a component, respectively.

Depending on a position of the proximal tibial cut 130 (FIG. 1) made, a varus/valgus angle 214, extension plane angle 216, external rotation 218, or joint extension gap can be affected. Similarly, a position of the distal femoral cut 128 (FIG. 1) can affect the location of the joint line 122, the extension gap, the varus/valgus angle 214, or the extension plane angle 216.

As used herein, “proximal” refers to a direction generally toward the torso of a patient, and “distal” refers to the opposite direction of proximal, i.e., away from the torso of a patient. “Anterior” refers to a direction generally toward the front of the patient, and “posterior” refers to the opposite direction of anterior, i.e., toward the rear of the patient. The term “anterior-posterior” can be anterior to posterior or posterior to anterior. The term “proximal-distal” can be proximal to distal or distal to proximal. The term “medial-lateral” can be lateral to medial or medial to lateral.

Revision knee surgery includes removing a previous implant and placing a new implant. In revision knee surgery, the native joint line cannot be re-created due to the removal of the underlying bone during a primary knee procedure. Similarly, bone and soft tissue landmarks may be lacking due to the removal of the underlying bone. The prostheses, systems and methods described herein can be useful in the revision surgical process including in ascertaining gap(s) such as the flexion and/or extension gaps and spacing to aid the physician in reconstructing the anatomy of the patient including the native joint line, femoral rotation, medial-lateral position and other anatomically useful information.

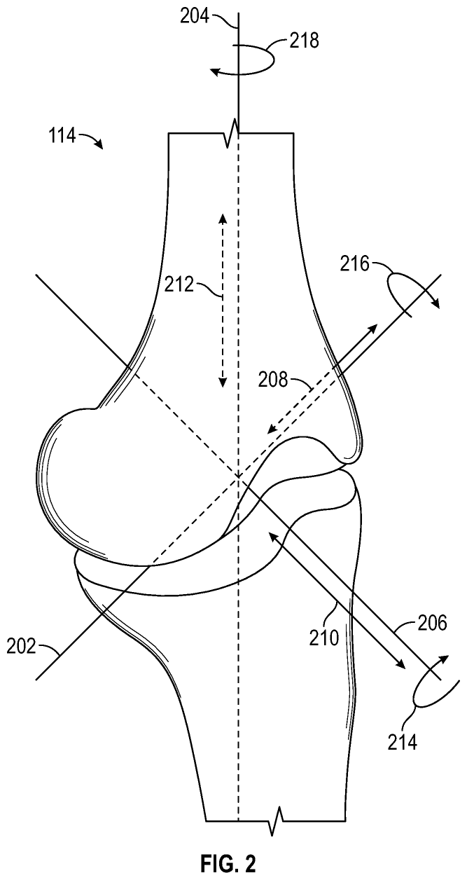

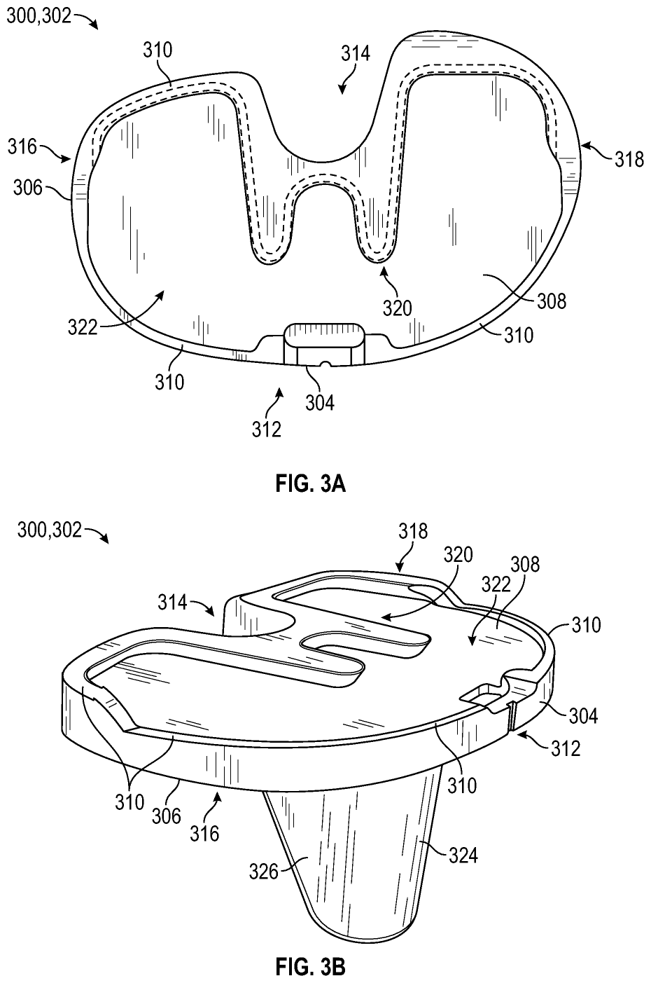

FIGS. 3A and 3B show an example of a tibial component 300 used as part of a system 302. The tibial component 300 can be a provisional tibial component (for temporary use in sizing and other activities during the surgery) or can be a permanent tibial implant.

FIGS. 3A and 3B show the tibial component 300 with the construction of a Persona® Total Knee Implant, manufactured by Zimmer Biomet, Inc. of Warsaw, Indiana. However, the construction of the tibial component 300 is purely exemplary and the tibial component 300 can have other constructs such as those of other commercial provisional tibial components or tibial knee implants such as those manufactured by Zimmer Biomet, Inc. The construct of the Persona® tibial component 300 shown in FIGS. 3A and 3B is further discussed in U.S. Pat. No. 8,628,580 B2, the entire disclosure of which is incorporated herein by reference.

FIGS. 3A and 3B show of the tibial component 300, which can include a periphery 304, a distal surface 306, a proximal surface 308 and a rail 310. The tibial component 300 can include an anterior side 312, a posterior side 314, a medial side 316 and a lateral side 318.

The periphery 304 can extend between the distal surface 306 and the proximal surface 308 and around all of the sides. The rail 310 can be positioned along the periphery 304 (thus can extend along any one or all of the anterior side 312, the posterior side 314, the medial side 316 and the lateral side 318) and can extend inward therefrom in some locations. The rail 310 can include along various extents coupling features such as bosses, notches, undercuts, dovetails, etc. for coupling with a polymer bearing component as known in the art. The rail 310 in the example of FIGS. 3A and 3B can include a two-pronged boss 320 as a coupling feature. This two-prong boss 320 can extend anteriorly forward from the posterior side 314 of the tibial component 300 and can be located in a central portion of the tibial component 300.

The distal surface 306 and/or the proximal surface 308 can be substantially flat. However, angulation of the distal surface 306 and/or the proximal surface 308 is contemplated in some examples. The distal surface 306 can be configured to seat on a resected proximal surface of the tibia 106 (see FIG. 1). The proximal surface 308 can be located generally opposite the distal surface 306. The proximal surface 308 can be located within the rail 310. Together the proximal surface 308 and the rail 310 can define a pocket 322. The pocket 322 can be a space within the rail 310 and extend to the proximal surface 308.

As shown in FIG. 3A, the tibial component 300 can include distal features such as a keel 324 and fin 326, used to stabilize and couple the tibial component 300 within a tibial canal. The keel 324 and the fin 326 (only one shown in FIG. 3A) can be coupled to the distal surface 306 and can extend distally therefrom.

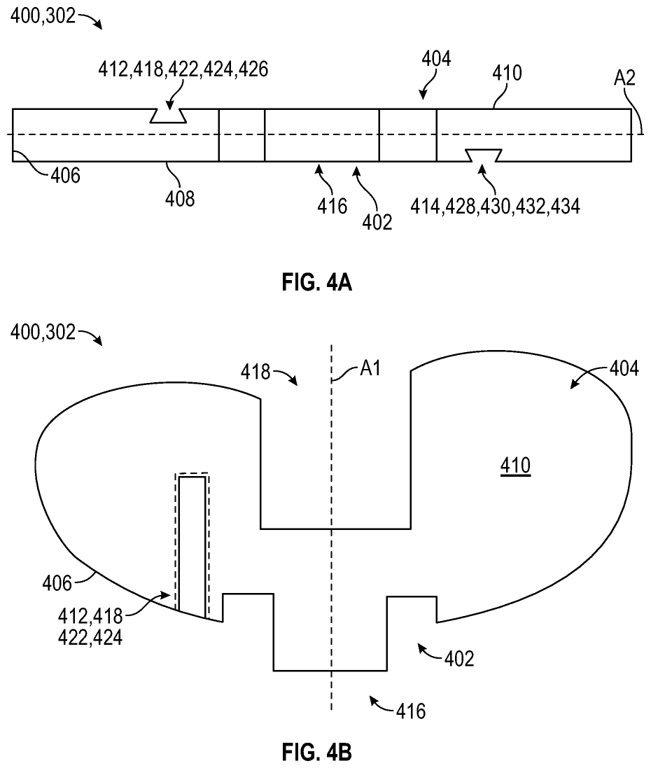

FIGS. 4A and 4B show plan views of a base component 400 that is part of the system 302. FIG. 4A shows primarily an anterior side 402 of the base component 400 and FIG. 4B shows primarily a proximal side 404 of the base component 400. The base component 400 can include a periphery 406, a distal surface 408, a proximal surface 410, a first feature 412, a second feature 414 (FIG. 4A) and a removal feature 416.

The periphery 406 can extend between the distal surface 408 and the proximal surface 410. The periphery 406 can be shaped to fit inside the pocket 322 (FIGS. 3A and 3B). Thus, the periphery 406 can be shaped to contact or be in close tolerance with an interior side of the rail 310 (FIGS. 3A and 3B). Thus, the periphery 406 can be smaller than the periphery 304 (FIGS. 4 and 3B) of the tibial component 300. The periphery 406 can have a cut-out 418 or other feature designed to accommodate the two-prong boss 320 (FIGS. 3A and 3B) of the tibial component 300 at a posterior side. It should be noted that the distal surface 408, and the base component 400 in general, can have no mechanical features (e.g., screws, screw holes, bosses, notches, undercuts, dovetails, etc. for mechanical coupling the base component 400 with the tibial component 300. Rather, the base component 400 can be set in place atop the proximal surface 308 (FIGS. 3A and 3B) of the tibial component 300 without any coupling therebetween and can be maintained is such position by a combination of friction, gravity, and in some cases, a compressive force (such as applied by the femur or a femoral provisional component).

The distal surface 408 can be substantially flat as shown. Alternatively, the distal surface 408 can be angled (e.g. varus-valgus, proximal-distal, anterior-posterior or in combination thereof) at a desired known angle, for example. The distal surface 408 can be configured to sit on the proximal surface 308 (FIGS. 3A and 3B) of the tibial component 300 in a stable manner.

The proximal surface 410 can be substantially flat as shown. Alternatively, the proximal surface 410 can be angled (e.g. varus-valgus, proximal-distal, anterior-posterior or in combination thereof) at a desired known angle for example.

The first feature 412 can comprise a notch 420 or other coupling feature as known in the art. The notch 420 can be recessed from the proximal surface 410. The notch 420 can have a dovetail 422 or other feature and can have an opening 424 to the proximal surface 410 and an opening 426 to the anterior side 402, for example. The second feature 414 can comprise a notch 428 or other coupling feature as known in the art. The notch 428 can be recessed from the distal surface 408. The notch 428 can have a dovetail 430 or other feature and can have an opening 432 to the distal surface 408 and an opening 434 to the anterior side 402, for example.

As shown in FIG. 4A, the first feature 412 and the second feature 414 can be identically shaped and sized. The first feature 412 and the second feature 414 can also be symmetrically arranged with respect to an anterior-posterior axis A1 (FIG. 4B) and a medial-lateral axis A2. This configuration for the first feature 412 and the second feature 414 allows the base component 400 simply to be flipped over (rotated 180 degrees about the axis A1) so as to be able to be utilized in both a right knee with a right knee provisional tibial component or right knee tibial implant and a left knee with a left knee provisional tibial component or left knee tibial implant. Put another way, the base component 400 is reversible in orientation such that the proximal surface 410 would become a distal surface and the distal surface 408 would become a proximal surface. Upon such reversal of orientation, the first feature 412 would then be at a lower right side to the viewer of FIG. 4A and the second feature 414 would be at an upper left side to the viewer of FIG. 4A.

The removal feature 416 can be at the anterior side 402 and can be a feature such as a projection that is graspable by fingers or a tool to remove the base component 400 from the pocket 322 (FIGS. 3A and 3B). The removal feature 416 can include notches that facilitate grasping of the projection. The removal feature 416 can extend from the periphery 304 (FIGS. 3A and 3B) of the tibial component 300 to facilitate grasping and removal, for example.

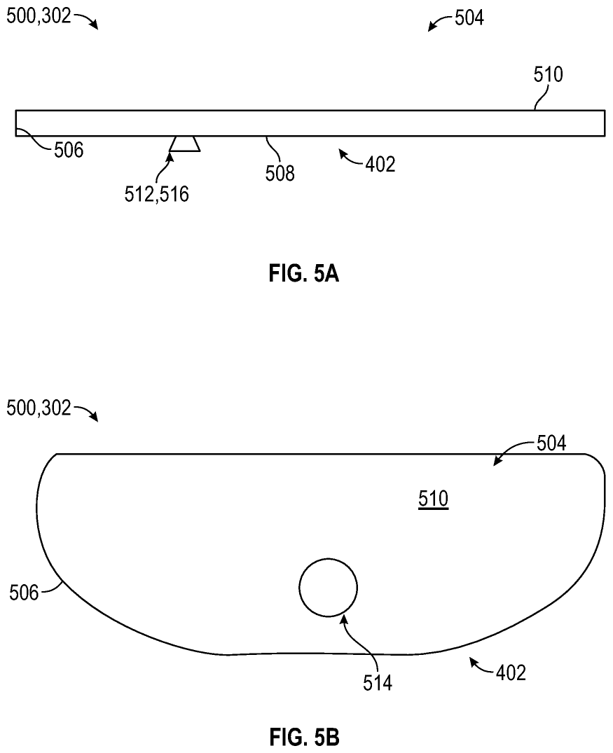

FIGS. 5A and 5B show plan views of a shim 500 that is part of the system 302. FIG. shows primarily an anterior side 502 of the shim 500 and FIG. 4B shows primarily a proximal side 504 of the shim 500. The shim 500 can include a periphery 506, a distal surface 508, a proximal surface 510, a feature 512 and a removal feature 514 (FIG. 5B).

The periphery 506 can extend between the distal surface 508 and the proximal surface 510. The periphery 506 can be generally shaped to fit within the knee space and not overhang or be recessed from the periphery 406 (FIGS. 4A and 4B) of the base component 400 by too great an extent. Thus, the periphery 506 can be shaped in any manner as long as the result is a stable arrangement of the shim 500 atop the base component 400 when coupled together and knee joint kinematics are not interfered with. The periphery 506 especially along a posterior side can be recessed so as to be set anteriorly back than the periphery 406 (FIGS. 4A and 4B) of the base component 400. This can provide space for the femur or femoral provisional to move through a range of motion.

The distal surface 508 can be substantially flat as shown. Alternatively, the distal surface 508 can be angled (e.g. varus-valgus, proximal-distal, anterior-posterior or in combination thereof) at a desired known angle for example. The distal surface 508 can be configured to sit on the proximal surface 410 (FIGS. 4A and 4B) of the base component 400 in a stable manner.

The proximal surface 510 can be substantially flat as shown. Alternatively, the proximal surface 510 can be angled (e.g. varus-valgus, proximal-distal, anterior-posterior or in combination thereof) at a desired known angle for example.

The feature 512 can comprise a projection 516 configured to couple by mating with the notch 420 (FIG. 4A) or notch 428 (FIG. 4A) of the base component 400 depending upon the orientation of the base component 400 as dictated by the knee the system 302 is being used in. The projection 516 can extend from the distal surface 508 a distance. The projection 516 can be designed to be received in the notch 420 or the notch 428. This coupling arrangement can be accomplished by aligning the projection 516 with the notch 420 or the notch 428 and sliding the shim 500 anterior-posterior onto and over the base component 400 such that the projection 516 is received in the notch 420 or the notch 428 and the distal surface 508 abuts the proximal surface 410 of the base component 400. Although the use of complementary mating features is shown in example of FIGS. 4A-5B, in some cases the shim 500 and base component 400 may not utilize mechanical coupling features. Rather, the shim 500 could simply be set on the base component 400 and could be maintained in such position by a combination of friction, gravity, and in some cases, a compressive force (such as applied by a femur or femoral provisional component).

The removal feature 514 can be an opening such as an aperture in the shim 500 at the proximal surface 510 that is graspable by a finger(s) or tool to facilitate removal.

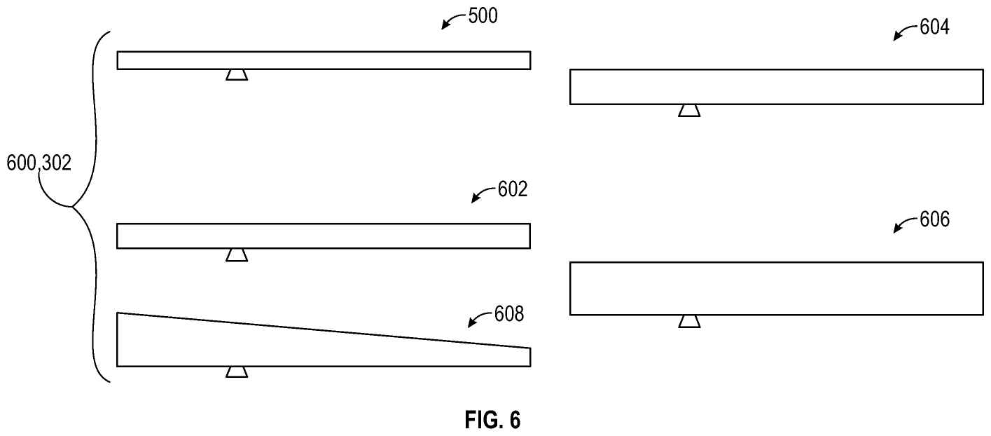

FIG. 6 shows the system 302 can include a set of shims 600 that includes the shim 500 of FIGS. 5A and 5B described previously. The set of shims 600 can include a plurality of shims 500, 602, 604, 606 and 608, for example. The construct of the shims shown is purely exemplary and can differ according to other examples. Each of the plurality of shims 500, 602, 604, 606 can have a different thickness as measured in a proximal-distal direction. Thus, for example, the shim 500 can be substantially 4 mm thick, the shim 602 can be substantially 8 mm thick as measured proximal-distal, the shim 604 can be substantially 12 mm thick as measured proximal-distal and the shim 606 can be substantially 16 mm thick as measured proximal-distal. The values provide above are purely exemplary and not intended to be limiting. The thicknesses of the shims can replicate the thicknesses of standard stock sizes of polymer tibial bearing component. The shim 608 can have a predefined angulation (e.g., between 1 degrees and 10 degrees) such as in the medial-lateral direction as shown in FIG. 6. However, the angulation of the shim 608 is purely exemplary and can be in another direction (anterior-posterior, proximal-distal or a combination thereof).

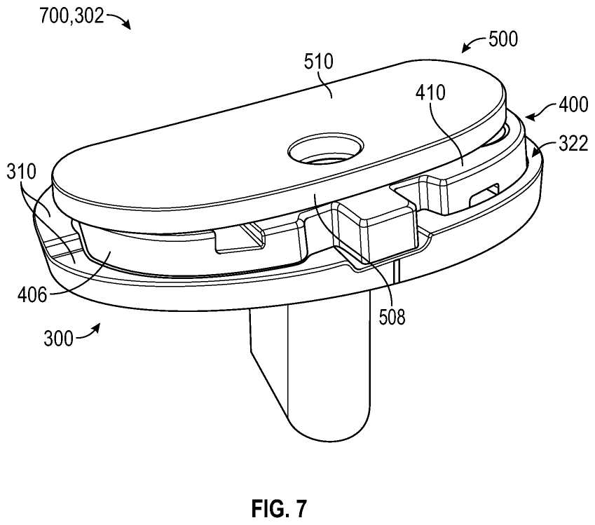

FIG. 7 shows a provisional tibial assembly 700 created using the system 302. The provisional tibial assembly 700 can include the tibial component 300, the base component 400 and the shim 500 as previously described.

The shim 500 may not be utilized in some examples where further proximal-distal height of the provisional tibial assembly 700 is not desired. Thus, the system 302 and the provisional tibial assembly 700 may comprise the tibial component 300 and the base component 400 with the shim 500 being optional.

In FIG. 7, the provisional tibial assembly 700 has the base component 400 seated within the pocket 322 defined by the rail 310 with the periphery 406 interfacing with an interior side of the rail 310. Thus, the base component 400 simply sits atop the proximal surface 308 (not shown in FIG. 7) of the tibial component 300. The provisional tibial assembly 700 has the shim 500 coupled with the base component 400 with the feature 512 (FIG. 5A) coupled with the first feature 412. In the coupled arrangement of FIG. 7, a distal surface 508 of the shim 500, which can be substantially flat, can abut and be atop the proximal surface 410 of the base component 400. The proximal surface 510 of the shim 500 (if used) can be substantially flat or can have a known angulation making the proximal surface 510 easy to reference off of. Furthermore, the relatively large surface area of the proximal surface 510 can be easy to place instruments on and to visualize with. Similarly, if the shim 500 is not utilized with the provisional tibial assembly 700, the proximal surface 410 of the base component 400 can be substantially flat or can have a known angulation. This can make the proximal surface 410 easy to reference off of. Furthermore, the relatively large surface area of the proximal surface 410 can be easy to place instruments on and to visualize with.

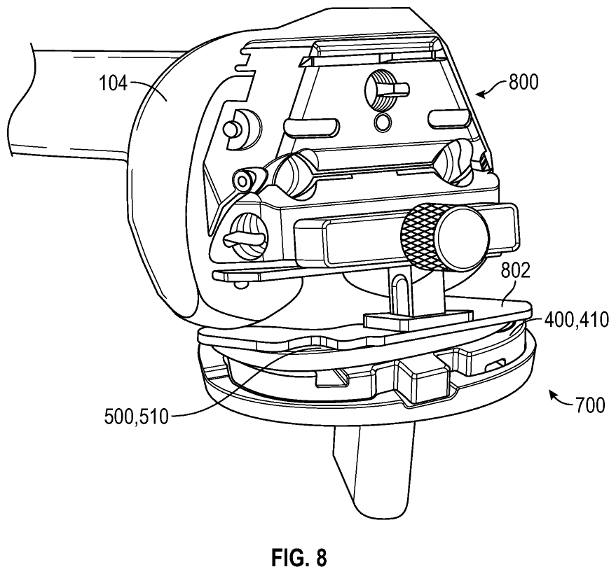

FIG. 8 shows a femoral cut guide 800 referencing off of the provisional tibial assembly 700, in particular, the proximal surface 510 of the shim 500 used as part of the provisional tibial assembly 700. The femoral cut guide 800 can include feet 802 or other features that can be set on the proximal surface 510 prior to one or more pins being used to couple the femoral cut guide 800 to the femur 104. The femoral cut guide 800 can be configured with slots or other features to guide one or more resects to the femur 104. The femoral cut guide 800 is just an example of one instrument that can use the proximal surface 510 or proximal surface 410 of the base component 400, which can be substantially flat, as a reference. Visual reference to the proximal surface 510 or proximal surface 410 can also be performed by the surgeon in checking gap(s) such as the flexion and/or extension gaps and spacing to aid the physician in reconstructing the anatomy of the patient including the native joint line, femoral rotation, medial-lateral position and other anatomically useful information.

It will be readily understood to those skilled in the art that various other changes in the details, material, and arrangements of the parts and method stages which have been described and illustrated in order to explain the nature of the inventive subject matter can be made without departing from the principles and scope of the inventive subject matter as expressed in the subjoined claims. For example, the order of method steps or stages can be altered from that described above, as would be appreciated by a person of skill in the art.

It will also be appreciated that the various dependent claims, examples, and the features set forth therein can be combined in different ways than presented above and/or in the initial claims. For instance, any feature(s) from the above examples can be shared with others of the described examples, and/or a feature(s) from a particular dependent claim may be shared with another dependent or independent claim, in combinations that would be understood by a person of skill in the art.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

The term “substantially”, “generally” or “about” means within 10% of the value provided. The term “flat” means having no angulation relative to at least one or a combination of the medial-lateral, proximal-distal and/or anterior-posterior directions. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A provisional tibial prosthesis system comprising:
a tibial component having a rail that defines a pocket and a distal surface configured to seat on a resected proximal surface of a tibia;
a base component configured to seat within the pocket of the tibial component when positioned thereon, wherein when so positioned with the pocket, the base component has a substantially flat proximal surface and a substantially flat distal surface; and
a shim having a feature configured to couple with a second feature of the base component when disposed thereon, wherein the shim has a substantially flat proximal surface.

2. The system of claim 1, wherein neither the proximal surface of the base component nor the proximal surface of the shim is configured to articulate with a femoral prosthesis.

3. The system of claim 1, wherein the feature of the shim is configured to mate with and be complementary to the second feature of the base component.

4. The system of claim 1, wherein the distal surface of the base component and the proximal surface of the base component have a substantially same shape and size so as to be reversable in orientation between a right knee and a left knee of a patient.

5. The system of claim 4, further comprising a third feature of the base component having an identical configuration with the second feature of the base component, wherein the third feature is on a distal side of the base component and the second feature is on a proximal side of the base component.

6. The system of claim 4, wherein the feature of the shim comprises a boss with a dovetail and the second feature of the base component comprises a notch with a corresponding dovetail.

7. The system of claim 1, wherein the shim is one of a plurality of shims each having a different proximal-distal thickness from one another.

8. The system of claim 7, wherein any one of the plurality of shims is stackable on the base component by sliding the feature of one of the plurality of shims anterior-posterior into the second feature of the base component.

9. The system of claim 1, wherein the feature and the second feature are complementary mating features configured to couple with one another in a mating engagement when the shim is placed atop the proximal surface of the base component.

10. The system of claim 9, wherein the feature comprises a male projection and the second feature comprises a complimentary female recess in the base component.

11. The system of claim 1, wherein each of the shim and the base component have a removal feature to aid in removing the shim and the base component from a knee joint.

12. The system of claim 1, wherein a periphery of the base component is configured to seat against an interior of the rail of the tibial component.

13. The system of claim 1, wherein the tibial component comprises one of a provisional component or a permanent implant.

14. The system of claim 1, further comprising a femoral cut guide configured to reference one of the proximal surface of the base component or the proximal surface of the shim when making a resection of a femur.

15. The system of claim 1, wherein the base component with the substantially flat distal surface rests on a substantially flat proximal surface of the tibial component within the pocket, and wherein the base component has no feature to couple with the tibial component when so seated on the substantially flat proximal surface of the tibial component but is held in position within the pocket atop the substantially flat proximal surface of the tibial component by gravity and/or a compressive force.

16. A provisional prosthesis system comprising:
a tibial component having a distal surface configured to seat on a resected proximal surface of a tibia;
a base component configured to mount on a tibial component and having a substantially flat proximal surface and a substantially flat distal surface; and
a set of shims, each of the set of shims configured to couple with the base component and having a different proximal-distal thickness, wherein each of the set of shims has a substantially flat proximal surface and a substantially flat distal surface.

17. The system of claim 16, wherein a periphery of the base component is configured to seat against an interior of a rail of the tibial component.

18. The system of claim 17, wherein the shim has a feature and the base component has a second, wherein the feature and the second feature are complementary mating features configured to couple with one another in a mating engagement when the shim is placed atop the proximal surface of the base component and further comprising a third feature of the base component having an identical configuration with the second feature of the base component, wherein the distal surface of the base component and the proximal surface of the base component have a substantially same shape and size so as to be reversable in orientation between a right knee and a left knee of a patient, and wherein the third feature is on a distal side of the base component and the second feature is on a proximal side of the base component.

19. A method of using a provisional tibial prosthesis system during knee arthroplasty procedure, the method comprising:
mounting a tibial component on a resected proximal surface of a tibia;
setting a base component with a substantially flat proximal surface on a proximal surface of the tibial component without mechanically coupling the base component to the tibial component with use of one or more coupling feature;
moving a knee joint through a range of motion; and
referencing at least one of a flexion gap and an extension gap relative to the substantially flat proximal surface of the base component.

20. The method of claim 19, further comprising:
coupling a shim having a substantially flat proximal surface to the base component such that the shim is set atop the substantially flat proximal surface of the base component;
moving the knee joint through a range of motion;
referencing at least one of a flexion gap and an extension gap relative to the substantially flat proximal surface of the shim.
setting a femoral cut guide atop one of the substantially flat proximal surface of the shim or the substantially flat proximal surface of the base component; and
performing a resection of a femur with the aid of the femoral cut guide.
